(19) United States
(12) Patent Application Publication
(10) Pub. No.: US 2008/0139765 A1
(43) Pub. Date: Jun. 12, 2008

(54) Polymers having improved cosmetic wear properties

(76) Inventors: Michael Spiegel, Caldwell, NJ (US); John Imperante, Califon, NJ (US); Anthony J. O' Lenick, Dacula, GA (US)

Correspondence Address: ANTHONY J. O'LENICK JR, 2170 LUKE EDWARDS RD, Dacula, GA, 30019, US

(21) Appl. No.: 11/807,867

(22) Filed: May 31, 2007

Related U.S. Application Data
(60) Provisional application No. 60/873,076, filed on Dec. 7, 2006.

(51) Int. Cl. A61K 31/78; C08F 20/06

(52) U.S. Cl. 526/220; 526/232.1; 526/318.5

(57) ABSTRACT

The present invention relates generally to improvements of wear resistant cosmetic compositions. Specifically, this invention relates to a cosmetic composition containing particular copolymers with glass transition temperatures greater than 70° Celsius that demonstrate enhanced wear in combination with a smooth, non-tacky feel. Since most carrier solvents formulated into cosmetics are weak solvents with high boiling points and slow evaporation rates the applied films remain wet and tacky resulting in poor cosmetic performance. The polymers of the present invention overcomes this deficiency due to the high Tg associate with the polymer which yields an apparent faster dry time in the applied coating thus improving the cosmetic's film integrity.

This application claims priority to and benefit of U.S. Provisional Application No. 60/873,076, filed Dec. 7, 2006, the disclosure of each of which are incorporated herein for all purposes.

### FIELD OF THE INVENTION

The present invention relates generally to improvements of wear resistant cosmetic compositions. Specifically, this invention relates to a cosmetic composition containing particular copolymers with glass transition temperatures greater than 70° Celsius that demonstrate enhanced wear in combination with a smooth, non-tacky feel. Since most carrier solvents formulated into cosmetics are weak solvents with high boiling points and slow evaporation rates the cosmetic films stay wet and tacky thus yielding an inferior product poor in cosmetic performance. The polymers of the present invention overcomes this deficiency due to the high Tg associate with the polymer which yields an apparent faster dry time in the applied coating thus improving the cosmetic's film integrity. A material's glass transition temperature, T_{g}, is the temperature below which molecules have little relative mobility. T_{g} is usually applicable to wholly or partially amorphous phases such as glasses and plastics. For inorganic or mineral glasses, such as common silicon dioxide (SiO₂) glass, it is the mid-point of a temperature range in which they gradually become more viscous and change from being liquid to solid. Thermoplastic (non-crosslinked) polymers are more complex because, in addition to a melting temperature, Tₘ, above which all their crystalline structure disappears, such plastics have a second, lower T_{g} below which they become rigid and brittle, and can crack and shatter under stress. Small molecular weight pure substances such as water have just one such condensed-phase temperature, below which they are solid crystals (or amorphous ice if cooled below T_{g} fast enough) and above which they are liquids. Above T_{g}, the secondary, non-covalent bonds between the polymer chains become weak in comparison to thermal motion, and the polymer becomes rubbery and capable of elastic or plastic deformation without fracture. This behavior is one of the things which make most plastics useful. But such behavior is not exhibited by crosslinked thermosetting plastics which, once cured, are set for life and will shatter rather than deform, never becoming plastic again when heated, nor melting.

### BACKGROUND OF THE INVENTION

Cosmetic coatings formulated with various organic waxes, solvents, polymers, additives and film formers that are known in the art to impart uniform films protective barriers can be compounded for the skin, hair and lips. The deleterious effects caused by the slow evaporation rate of the solvents typically utilized in cosmetic formulations result in slow dry time, poor adhesion and tend to delaminate off the surface to which they are applied. The tack and poor adhesion lead to product transfer which requires the consumer to reapply the product more frequently

Current products that reduce the dry time also have limitations. The key limitations are an objectionable tacky feel to the skin, lips or lashes on application and a tendency to pull against the skin when applied.

Accordingly, there is a need to develop a wear resistant product that can be applied to the hair, skin, lips or nails that will balance the dry time of the cosmetic while not compromising the film properties.

U.S. Pat. No. 6,083,516 issued Jul. 4, 2000 to Curtis, et al entitled Wear resistant cosmetics teaches that wear-resistant cosmetic products can be formulated in cosmetically acceptable carriers using polymers from about 0.1 weight percent to about 15 weight percent that incorporate an alkyl cycloalkylacrylate constituent incorporated on the polymer, preferably from about 0.1 weight percent to about 15 weight percent.

Since cosmetic products are not permanent coatings the solvents act more like a plasticizers than solvents. By increasing the glass transition temperature (Tg) or hardness of the polymer the apparent dry time is much faster. The quick “dry to touch” yields a tack free, durable temporary film. However, as the solvent continues to evaporate the coating will become more brittle and eventually fail. The failure should not be observed by the end user due to the short time for expected use.

### SUMMARY OF THE INVENTION

We have surprisingly found that using aliphatic acrylates and methacrylates specifically t-butyl methacrylate in combination with other monomers typically used in vinyl polymerization, polymers with Tg's greater then 70 degrees Celsius can be formulated for solubility and performance for use in the personal care products. Polymers that have been heretofore unavailable can be made. Such polymers are extremely soluble in weak solvents with low solubility constants or low Kb values. By varying the isomers, adjusting the carbon length and molecular weight it is possible to offer polymers that are soluble in cosmetic carriers, with fast dry time that do not have the limitations stated previously.

Against the foregoing background, it is a primary object of the present invention to provide a cosmetic composition having superior wear resistance.

Another object of the present invention to provide cosmetic compositions that produce quick drying protective films on the skin, hair or lips.

It is yet another object of the present invention to provide a wear resistant cosmetic composition that is neither tacky, dry nor drags against the skin, hair or lips upon application.

To the accomplishment of the foregoing objects and advantages, we have surprisingly found that the present invention directed to wear-resistant cosmetic composition must include tertiary butyl methacryaltes and C18-C22 acrylates and methacrylates. Multiple polymers were synthesized with various dry times, glass transition temperatures, flexibility and functionalities for improved performance.

The present invention is drawn to a process for making a compound conforming to the following structure:

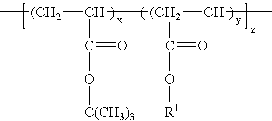

wherein;
R¹ is any other vinyl functional monomer used in vinyl synthesis;
x is an integer ranging from 3;
y is an integer greater than 3;
z is an integer ranging from 3 to 10,000;
said polymer has a T_{g} of greater than 70° Celsius.

The present invention is also drawn to a polymer prepared by reacting:
a first monomer conforming to the following structure CH₂═CH—C(O)—O—C(CH₃)₃
with a second monomer conforming to the following structure CH₂═CH—C(O)—OR¹
Wherein the polymer is soluble in cosmetic carriers and has a Tg greater than 70° C. wherein;
   R¹ is alkyl chain having 1 to 22 carbon atoms.

In a preferred embodiment the present invention is also drawn to a polymer prepared by reacting: a first monomer conforming to the following structure CH₂═CH—C(O)—O—C(CH₃)₃ with a second monomer conforming to the following structure CH₂═CH—C(O)—OR¹ to be soluble in characteristic cosmetic solvents and having a Tg greater than 70° Celsius;

wherein;

### R¹ is alkyl having 1 to 22 carbon atoms,

wherein said reaction is a free radical reaction conducted in the presence of a free radical initiator selected from the group consisting of t-amyl peroctoate, benzoyl peroxide, azobisisobutrylnitrile and mixtures thereof.

### DESCRIPTION OF THE PREFERRED EMBODIMENTS

The present invention provides a wear-resistant cosmetic such as, for example, a lipstick, a makeup, a sunscreen, blush or mascara. It has surprisingly been found that a polymer of the present invention provides superior wear resistance to such cosmetic compositions or formulas, both alone and preferably in combination with an alkyl cycloalkylacrylate copolymer. Moreover, it has surprisingly been found that cosmetic formulas containing the compounds of the present invention are non-tacky and have a pleasant feel when applied to the skin. These compositions are easy to apply, have improved skin and lip adherence (transfer resistance), and provide enhanced wear and longevity.

Most preferably, the wear-resistant cosmetic composition according to the present invention includes a polymer of the present invention in a cosmetically acceptable carrier. In a preferred embodiment the ratio of the first monomer to the second monomer ranges from 1:10 to 1:1. In a more preferred embodiment the ratio of the first monomer to the second monomer ranges from 1:10 to 1:2.

The reaction is a free radical reaction conducted in the presence of a free radical initiator selected from the group consisting of t-amyl peroctoate, benzoyl peroxide, azobisisobutrylnitrile and mixtures thereof.

### EXAMPLES

Polymers of the Present Invention

The polymers of the present invention are prepared by reacting acrylate monomers, one having an alkyl group C18-C22, and the other having a tertiary butyl carbon arrangement. This is very important for the properties of the product. Additionally a cosmetically acceptable solvent is used. By cosmetically acceptable is meant the solvent has no mal odor. Finally a free radical initiator is added. Free radical imitators are well known in the art. Preferred initiators are selected from the group consisting of t-amyl peroctoate, benzoyl peroxide, azobisisobutrylnitrile and mixtures thereof.

### Example 1

| Ingredient | Weight % |
|---|---|
| Permethyl 99 | 48.0 |
| T-butyl methacrylate | 41.0 |
| Behenyl methacrylate | 10.0 |
| T-amyl peroctoate | 1.0 |

### The contents were added to a reaction vessel, heated to 250° F. and reacted to greater than 99.8% conversion.

### Example 2

| Ingredient | Weight % |
|---|---|
| Permethyl 99 | 48.0 |
| T-butyl methacrylate | 21.0 |
| Iso-butyl methacrylate | 20.0 |
| Behenyl methacrylate | 10.0 |
| t-amyl peroctoate | 1.0 |

### The contents were added to a reaction vessel, heated to 250° F. and reacted to greater than 99.8% conversion.

### Example 3

| Ingredient | Weight % |
|---|---|
| Permethyl 99 | 48.0 |
| iso-butyl methacrylate | 41.0 |
| Behenyl methacrylate | 10.0 |
| t-amyl peroctoate | 1.0 |

### The contents were added to a reaction vessel, heated to 250° F. and reacted to greater than 99.8% conversion.

### Example 4

| Ingredient | Weight % |
|---|---|
| Permethyl 99 | 48.0 |
| T-butyl methacrylate | 41.0 |
| Stearyl methacrylate | 10.0 |
| t-amyl peroctoate | 1.0 |

### The contents were added to a reaction vessel, heated to 250° F. and reacted to greater than 99.8% conversion.

### Example 5

| Ingredient | Weight % |
|---|---|
| Permethyl 99 | 48.0 |
| T-butyl methacrylate | 41.0 |
| Behenyl methacrylate | 10.0 |
| Vazo 67 | 1.0 |

### The contents were added to a reaction vessel, heated to 250° F. and reacted to greater than 99.8% conversion.

### Example 6

| Ingredient | Weight % |
|---|---|
| Permethyl 99 | 48.0 |
| T-butyl methacrylate | 41.0 |
| Behenyl methacrylate | 10.0 |
| Benzoyl Peroxide | 1.0 |

### The contents were added to a reaction vessel, heated to 210° F. and reacted to greater than 99.8% conversion.

### Example 7

| Ingredient | Weight % |
|---|---|
| IN-2 Isononyl Isononoate | 48.0 |
| T-butyl methacrylate | 41.0 |
| Behenyl methacrylate | 10.0 |
| Benzoyl Peroxide | 1.0 |

### The contents were added to a reaction vessel, heated to 210° F. and reacted to greater than 99.8% conversion.

### Example 8

| Ingredient | Weight % |
|---|---|
| Permethyl 99 | 28.0 |
| T-butyl methacrylate | 66.0 |
| Behenyl methacrylate | 5.0 |
| t-amyl peroctoate | 1.0 |

### The contents were added to a reaction vessel, heated to 250° F. and reacted to greater than 99.8% conversion.

### Example 9

| Ingredient | Weight % |
|---|---|
| Permethyl 99 | 48.0 |
| T-butyl methacrylate | 1.0 |
| Iso-butyl methacrylate | 30.0 |
| Behenyl methacrylate | 20.0 |
| t-amyl peroctoate | 1.0 |

### The contents were added to a reaction vessel, heated to 250° F. and reacted to greater than 99.8% conversion.

### Example 10

| Ingredient | Weight % |
|---|---|
| Permethyl 99 | 48.0 |
| iso-butyl methacrylate | 11.0 |
| Behenyl methacrylate | 40.0 |
| t-amyl peroctoate | 1.0 |

### The contents were added to a reaction vessel, heated to 250° F. and reacted to greater than 99.8% conversion.

### Example 11

| Ingredient | Weight % |
|---|---|
| Permethyl 99 | 48.0 |
| T-butyl methacrylate | 11.0 |
| Stearyl methacrylate | 40.0 |
| t-amyl peroctoate | 1.0 |

### The contents were added to a reaction vessel, heated to 250° F. and reacted to greater than 99.8% conversion.

### Example 12

| Ingredient | Weight % |
|---|---|
| Permethyl 99 | 48.0 |
| T-butyl methacrylate | 41.0 |
| Behenyl methacrylate | 10.0 |
| Vazo 67 | 1.0 |

### The contents were added to a reaction vessel, heated to 250° F. and reacted to greater than 99.8% conversion.

### Example 13

| Ingredient | Weight % |
|---|---|
| Permethyl 99 | 48.0 |
| T-butyl methacrylate | 11.0 |
| Behenyl methacrylate | 40.0 |
| Benzoyl Peroxide | 1.0 |

### The contents were added to a reaction vessel, heated to 210° F. and reacted to greater than 99.8% conversion.

### Example 14

| Ingredient | Weight % |
|---|---|
| IN-2 Isononyl Isononoate | 48.0 |
| T-butyl methacrylate | 21.0 |
| Behenyl methacrylate | 30.0 |
| Benzoyl Peroxide | 1.0 |

### The contents were added to a reaction vessel, heated to 210° F. and reacted to greater than 99.8% conversion.

### The residual monomers were determined by Gas Chromatography/Mass Spectroscopy.

### All polymers had residual monomer levels below 2000 ppm.

### APPLICATIONS EXAMPLES

### Mascara

### PTC3-63/1

| | Trade Name | INCI Name | Company | Percent |
|---|---|---|---|---|
| A | Pelemol GTB | Tribehenin | Phoenix | 8.00 |
| | | | Chemical | |
| A | Pelemol H S A | Hydroxystearic Acid | Phoenix | 6.00 |
| | | | Chemical | |
| A | Stearic Acid | Stearic Acid | House | 10.00 |
| A | Beeswax 422P | Beeswax | S&P | 6.00 |
| A | Pelemol D2000 | Polypropylene glycol 2000 Dimer | Phoenix | 1.00 |
| | | Dinoleate | Chemical | |
| A | Pelemol MS-4 | Isododecane acrylates copolymer | Phoenix | 10.00 |
| | | | Chemical | |
| | Phenomulse CE-1 | Polyhydroxystearic Acid, Isononyl | Phoenix | 8.00 |
| | | Isononanoate, Ethylhexyl | Chemical | |
| | | Isononanoate, Sodium | | |
| | | Cocamidopropyl PG-Dimonium | | |
| | | Chloride Phosphate/Methy | | |
| | | Perflurobutyl Ether Methyl | | |
| | | Perfluoroisobutyl Ether | | |
| A | Germaben II | Propylene Glycol, Diazolidinyl Urea | ISP | 1.00 |
| | | Methyl Paraben Propyl Paraben | | |
| B | Distilled Water | Water | House | 30.50 |
| B | Keltrol F | Xanthan Gum | CP Kelco | 0.30 |
| C | 33-5198 | Iron Oxide | Sun Chemical | 10.00 |
| C | Arlacel 83 | Sorbitan Sesquioleate | Uniqema | 0.50 |
| D | Distilled Water | Water | | 4.70 |
| D | TEA | Triethanolamine 99% | House | 4.00 |
| | | | | 100.00 |

Procedure:
Heat phase B to 80oC with homomixing once dispersed add phase C & D and homomix
to disperse pigment. Heat phase A to 85oC then add phase C to Phase AB slowly.
Homomix for 5 minutes and switch to sweep. Add phase E and cool to 35oC.

### Sunscreen SPF 30

### PTC3-70/1

| | Trade Name | CTFA Name | Supplier | Percent |
|---|---|---|---|---|
| A | Distilled Water | Water | House | 40.15 |
| A | Butylene Glycol | Butylene Glycol | House | 2.00 |
| A | Allantoin | Allantoin | House | 0.10 |
| A | Versene Na | Disodium EDTA | Dow Chemical | 0.20 |
| A | Methyl Paraben | Methyl Paraben | House | 0.30 |
| B | Pecosil P-49 | Pentaerythrityl | Phoenix Chemical | 7.00 |
| | | Tetraisononanoate | | |
| B | Arlacel 165 | Glyceryl Stearate, PEG-100 | Uniqema | 8.00 |
| | | Stearate | | |
| B | DC 200/10cs | Dimethicone | Dow Corning | 3.00 |
| B | Lanette O | Cetearyl Alcohol | Cognis | 2.00 |
| B | Pelemol II | Isostearyl Isostearate | Phoenix Chemical | 2.00 |
| B | Tween 60 | Polysorbate 60 | Uniqema | 0.80 |
| B | Propyl Paraben | Propyl Paraben | House | 0.20 |
| C | DC65ZCI | Zinc Oxide, Cyclomethicone | Kobo | 4.00 |
| | | PEG/PPG-18/18 Dimethicone | | |
| | | Dimethicone | | |
| C | DC45TS | Titanium DiOxide Cyclomethicone | Kobo | 5.00 |
| | | Polyglyceryl-6 Polyricinoleate | | |
| | | Stearic Acid Aluminum Hydroxide | | |
| C | Parsol MCX | Ethylhexyl Methoxycinnamate | Roche | 5.45 |
| C | Uvinol M-40 | Benzophenone-3 | BASF | 4.00 |
| C | Escalol 587 | Ethylhexyl Salicylate | ISP | 4.00 |
| C | Escalol 597 | Octacrylene | ISP | 5.00 |
| D | Giovarez MS-4 | Isododecane acrylates | Phoenix Chemical | 6.00 |
| | | copolymer | | |
| E | Germaben II | Propylene Glycol Diazolidinyl | ISP | 0.80 |
| | | Urea Methyl Paraben Propyle | | |
| | | Paraben | | |
| | | | | 100.00 |

Procedure:
Heat phase A to 75-80° C. Heat phase B in separate vessel to 75-89° C.
Premix phase C and mix until uniform. (homomix) Once uniform add phase C to phase B and phase D to phase BC. Maintain temp of phase BCD of 75-80° C.
Add phase BCD to Phase A with homomixing. Mix 5 minutes. Begin sweep mixing and cool batch to 50° C. Add phase E to batch. Cool to 30-35° C. drop batch.

### Phoenix Chemical

### Transferproof Leg Make-up

### PTC3-71/1

| | Trade Name | INCI Name | Company | Percent |
|---|---|---|---|---|
| A | Water | Di Water | House | 35.00 |
| A | NaCl | NaCl | House | 2.00 |
| A | Hampene Na3 | Na3EDTA | House | 0.05 |
| A | Glycerine | Glycerine | House | 3.00 |
| A | BG | Butylene Glycol | House | 3.00 |
| A | Germizide PSB | Phenoxyethanol, Chlorphenesin, Benzoic Acid, Butylene | Engelhard | 1.00 |
| | | Glycol | | |
| B | Pecosil G-5 | Dimethicone/Isododecane/Phenyl Trimethicone/ | Phoenix | 3.00 |
| | | Trimethylsiloxysilicate/C30-45 Olefin | Chemical | |
| B | Phoenomulse CE-I | Polyhydroxystearic Acid, Isononyl Isononanoate, | Phoenix | 9.45 |
| | | Ethylhexyl Isononanoate, Sodium Cocamidopropyl PG- | Chemical | |
| | | Dimonium Chloride Phosphate/Methy Perflurobutyl | | |
| | | Ether Methyl Perfluoroisobutyl Ether | | |
| B | DC45TS | Titanium DiOxide Cyclomethicone Polyglyceryl-6 | Kobo | 6.00 |
| | | Rolynicinoleate Stearic Acid Aluminum Hydroxide | | |
| B | WE70U | Titanium Dioxide, Polyglyceryl-4, Cetyl PEG/Ppg-10/1 | Kobo | 5.00 |
| | | Dimethicone, Hexyl Laurate and Isopropyl Titanium | | |
| | | Triisostearate | | |
| B | WE70R | Iron Oxide, Polyglyceryl-4, Cetyl PEG/Ppg-10/1 | Kobo | 0.40 |
| | | Dimethicone, Hexyl Laurate and Isopropyl Titanium | | |
| | | Triisostearate | | |
| B | WE55Y | Iron Oxide, Polyglyceryl-4, Cetyl PEG/Ppg-10/1 | Kobo | 1.40 |
| | | Dimethicone, Hexyl Laurate and Isopropyl Titanium | | |
| | | Triisostearate | | |
| B | WE70B | Titanium Dioxide, Polyglyceryl-4, Cetyl PEG/Ppg-10/1 | Kobo | 0.20 |
| | | Dimethicone, Hexyl Laurate and Isopropyl Titanium | | |
| | | Triisostearate | | |
| B | Mica AS | Mica & Triethoxycaprylylsilane | LCW | 3.00 |
| B | Pecosil DCF 1818 | Cyclomethicone & PEG/PPG-18/18 Dimethicone | Phoenix | 16.30 |
| | | | Chemical | |
| B | Giovarez MS-4 | Isododecane acrylates copolymer | Phoenix | 6.00 |
| | | | Chemical | |
| B | Pecosil DB | Dimethiconal Behenate | Phoenix | 3.20 |
| | | | Chemical | |
| B | Pelemol P-49 | Pentaerythrityl Tetraisononanoate | Phoenix | 2.00 |
| | | | Chemical | |
| | | | | 100.00 |

Procedure:
Heat phase B to 50 degrees C. with homomixing. Heat phase A to 50 degrees C. Slowly add phase A to phase B with homomixing for 5 minutes.
Drop batch at 35 degrees C.

### Transferproof Lipstick

### PTC3-73/1

| | Trade Name | INCI Name | Company | Percent |
|---|---|---|---|---|
| A | Synthic wax 170 | Polyethylene | S&P | 7.00 |
| A | Ceresin 1022 | Ceresin | S&P | 3.00 |
| A | Ozokerite 1020P | Oxokerite | S&P | 2.00 |
| A | Paraffin SP-674 | Paraffin | S&P | 2.00 |
| A | Pelemol H S A | Hydroxystearic Acid | Phoenix | 2.00 |
| | | | Chemical | |
| A | DC 5562 Carbinol Fluid | Bis-Hydroxyethoxypropyl Dimethicone | Dow Corning | 1.00 |
| A | Pelemol TGC | Trioctyldodecyl Citrite | Phoenix | 4.50 |
| | | | Chemical | |
| A | Vit. E | Tocopherol acetate | Rohm & Haas | 0.10 |
| A | Pelemol P-49 | Pentaerythrityl Tetraisononanoate | Phoenix | 2.05 |
| | | | Chemical | |
| A | Pecosil G | Dimethicone, Cyclomethicone, Phenyl | Phoenix | 20.00 |
| | | Trimethicone, Trimethylsiloxysilicate | Chemical | |
| A | Pelemol D-2000 | Polypropylene glycol 2000 Dimer Dinoleate | Phoenix | 2.30 |
| | | | Chemical | |
| A | Pelemol DP-144B | Dipentaerythrityl Tetrabehenate | Phoenix | 4.00 |
| | | Polyhydroxystearate | Chemical | |
| B | Plearlglo UVR | Bismuth Oxychloride | Phoenix | 4.00 |
| | | | Chemical | |
| B | Silk Mica | Mica | Rona | 2.65 |
| C | DC345 | Cyclomethicone | Dow Corning | 15.10 |
| C | Giovarez MS-4 | Isododecane acrylates copolymer | Phoenix | 10.00 |
| | | | Chemical | |
| C | PhoenoMulse CE-1 | Polyhydroxystearic Acid, Isononyl | Phoenix | 8.00 |
| | | Isononanoate, Ethylhexyl Isononanoate, | Chemical | |
| | | Sodium Cocamidopropyl PG-Dimonium | | |
| | | Chloride Phosphate/Methy Perflurobutyl | | |
| | | Ether Methyl Perfluoroisobutyl Ether | | |
| D | Red 7 19-011 | FD&C Red 7 | Sun | 1.10 |
| D | Red I/O | Iron Oxide | Sun | 1.55 |
| D | Black I/O | Iron Oxide | Sun | 0.20 |
| D | Brown 33-115 | Iron Oxide | Sun | 0.75 |
| D | TiO2 | Titanium Dioxide | Sun | 1.00 |
| D | yellow 5 | FD&C Yellow 5 | Sun | 0.20 |
| E | MP-29 | TiO₂, Mica, Iron Oxides | Rona | 5.50 |
| | | | | 100.00 |

Procedure:
Heat phase A to 80-85° C. Once clear add phase B and Phase D
Rollermill 3× to disperse pigments. Once pigments are ground heat back to 80-85° C. add phase C and phase E. Cool to 65-70° C. and pour samples. Keep batch covered as much as possible to minimize evaporation.

The compounds of the present invention provide cosmetic compositions that demonstrate enhanced wear in combination with a smooth, non-tacky feel. Since most carrier solvents formulated into cosmetics are high boiling, slow evaporating solvents, the resulting films remain wet and tacky resulting in poor cosmetic performance. The polymers of the present invention overcome this deficiency by making use of polymers with high glass transition temperature to compensate for the long evaporation time of the solvent.

While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth hereinabove but rather that the claims be construed as encompassing all the features of patentable novelty which reside in the present invention, including all features which would be treated as equivalents thereof by those skilled in the art to which the invention pertains.

1. A polymer prepared by reacting:
(a) a first monomer conforming to the following structure CH₂═CH—C(O)—OR¹ with
(b) a second monomer conforming to the following structure CH₂═CH—C(O)—O—C(CH₃)₃
wherein;
R¹ is alkyl having to 6-22 carbon atoms;
The ratio of the first monomer to the second monomer ranges from 1:10 to 10:1.

2. A polymer of claim 1 wherein the ratio of the first monomer to the second monomer ranges from 1:10 to 1:1.

3. A polymer of claim 1 wherein the ratio of the first monomer to the second monomer ranges from 1:10 to 1:2.

4. A polymer of claim 1 wherein said reaction is a free radical reaction conducted in the presence of a free radical catalyst selected from the group consisting of t-amyl peroctoate, benzoyl peroxide, azobisisobutrylnitrile and mixtures thereof.
